(19) United States
(12) Patent Application Publication
(10) Pub. No.: US 2010/0272814 A1
(43) Pub. Date: Oct. 28, 2010

(54) METHOD AND MEANS FOR PRODUCING BRONCHORELAXATION

(75) Inventor: Staffan Skogvall, Lund (SE)

Correspondence Address: DICKSTEIN SHAPIRO LLP, 1633 Broadway, NEW YORK, NY, 10019, US

(73) Assignee: Pharmalundensis AB, Lund (SE)

(21) Appl. No.: 12/747,337

(22) PCT Filed: Dec. 15, 2008
(86) PCT No.: PCT/SE08/00700

§ 371 Date: Jun. 10, 2010

(30) Foreign Application Priority Data
Dec. 19, 2007 (SE) 0702824-4

(51) Int. Cl. A61K 9/14; A61K 31/795; A61K 31/745; A61K 31/197; A61K 31/19; A61K 31/095; A61K 31/10; A61K 33/18; A61K 33/00; A61P 11/08; A61P 11/06; A61K 31/765; A61K 31/74

(52) U.S. Cl. 424/489; 424/78.35; 424/78.1; 514/566; 514/574; 514/561; 514/706; 514/711; 424/667; 424/723; 424/78.37; 424/78.31

(57) ABSTRACT

A method of producing bronchorelaxation in the lungs of a human or animal affected by airway obstruction comprises administration of a pharmacologically effective amount to the body or to the intestine of said human or animal of an agent capable of binding mercury in elemental, ionic and/or organic form present in the body or in the intestinal lumen to enhance its excretion via the feces and/or the urine. A corresponding use of the agent and its use for the manufacture of a medicament are also disclosed.

### FIELD OF THE INVENTION

The present invention relates to a method and a means for producing bronchorelaxation in the airways of a human or an animal. The present invention also relates to a method and means for treating chronic obstructive pulmonary disease and asthma in a human or an animal, and to corresponding uses.

### BACKGROUND OF THE INVENTION

Mercury exists in a number of forms, all being more or less toxic. The major forms of mercury humans are exposed to mercury vapor, Hg⁰, and methylmercury compounds (Clarkson, 1997). Mercury vapor is released naturally from volcanic activity and anthropogenic from mining of cinnabar, burning of coal, from chloro-alkali industry, municipal waste incinerators, crematoria and dental offices. Other sources of mercury include amalgam tooth fillings, vaccinations, batteries, paint etc. It has also been described that cigarettes release a substantial amount of inorganic mercury, estimated to around 5-7 ng per cigarette (Suzuki et al., 1976).

Atmospheric mercury vapor is slowly transformed to a water-soluble form and returned to the earth's surface in rain. The mercury can be methylated by micro-organisms to mono-methylmercury. This enters the aquatic food chain, bio-accumulates and becomes the predominant source of organic mercury in humans.

Major toxicological effects of inhaled elemental mercury include tremor, erethism (i.e. shyness, hallucinations or aggressive behavior) and gingivitis, collectively called mercurialism. Symptoms caused by methylmercury include skin paresthesia, ataxia, constriction of the visual field, paralysis and death (Clarkson, 2002). Recently it has been suggested that methylmercury can increase the risk of cardiovascular disease (Virtanen et al., 2007).

Methylmercury is well absorbed from food (about 95% of the oral intake) (Clarkson, 1997) and taken up by all tissues. The half time in whole body is slow, and has been reported to be around 70 days. Elimination takes place mainly by the fecal route. Methylmercury is secreted in the bile, possibly as cysteine complexes (Norseth and Clarkson, 1971), but is almost completely reabsorbed lower down in the intestinal tract, thus demonstrating an enterohepatic recirculation (Norseth and Clarkson, 1971; Clarkson et al., 1973). However, a small fraction of the methylmercury is converted to inorganic mercury by the intestinal microflora, and is subsequently excreted in the feces. In contrast to the readily absorbed organic mercury compounds, mercury salts are poorly absorbed from the intestine, with only about 7% absorption of mercuric chloride from an oral dose.

Several substances have been used to bind various forms of mercury. EDTA (ethylenediaminetetraacetic acid), BAL (British anti-Lewisite or 2,3-dimercapto-1-propanol), D-penicillamine, DMSA (meso-2,3-dimercaptosuccinic acid) and DMPS (2,3-dimercaptopropane-1-sulfonate) have all been used to treat humans exposed to elemental or inorganic mercury. They are chelating agents and most of them form chelates that mainly are excreted in the urine.

Methylmercury is probably not chelated since the methylmercury cation only forms one bond to a chelating agent. The methylmercury cation forms instead a thermodynamically stable bond with a deprotonated thiol group (Goyer et al., 1995).

Other methods have been developed to increase the excretion of methylmercury compounds. Mice subjected to food containing methylmercury chloride and a polystyrene resin, containing fixed sulfhydryl (thiol) groups, doubled their rate of excretion of methylmercury, compared to the control group not subjected to the polystyrene resin (Clarkson et al., 1973).

Thiol and thioether functional groups have been found to also react with other metal ions than those of mercury and in order to increase selectivity ureasulfonamide polymers have been used for mercury ion uptake from contaminated water (Senkal and Yavuz, 2006). Dithiocarbamate-incorporated monosize polystyrene microspheres have been tested for adsorption of mercury(II) ions in wastewater (Denizli et al., 2000). Bicine (N,N-bis(2-hydroxy-ethyl)glycine) on macroporous polystyrene divinyl benzene have also been tested for adsorption of mercury(II) ions from water (Dev and Rao, 1995). These adsorption studies have however all been on aqueous solutions and not for treating human mercury poisoning.

Activated charcoal is used in medical applications to treat poisoning and oral overdoses of various medicaments. Activated charcoal has a very large surface area; 1 gram has a surface area of 300-2000 m² (Greenwood et al., 1984).

Impregnated activated charcoals are carbonaceous adsorbents which have chemicals finely distributed on their internal surface. The impregnation optimizes the existing properties of the activated charcoal giving a synergism between the chemicals and the charcoal (Carbo Tech-Aktivkohlen GmbH, Germany). Iodinated activated charcoal has been used for many years to bind mercury in various types of gas.

Chronic obstructive pulmonary disease (COPD) and asthma are important causes of morbidity, mortality and health-care costs worldwide. The estimated prevalence of COPD in many western countries is more than 10% of the population (Mannino and Buist, 2007). In the USA, COPD was the primary reason for hospital discharge 9.8 million times and a secondary reason for discharge an additional 37.5 million times from 1979 to 2001. COPD is estimated to cause more than 80,000 deaths every year in the USA. It has been estimated that the total national cost in the USA for COPD was US$ 32.1 billion for the year 2003.

Approximately 300 million people worldwide currently have asthma. Most are found in the industrialized countries, which have an asthma prevalence of ˜10% in adults and almost 20% in children. The rate of emergency hospital admissions during the early 2000 was 10/100,000 each year in adults and 100/100,000 in young children in the UK (Anderson et al., 2007). Asthma causes 1,200 deaths each year in the United Kingdom alone. The financial burden of patients with asthma in different western countries amounts to around US$ 300 billion each year.

COPD is associated with tobacco smoking and is characterized by inflammation in the airways and a gradual decline in lung function. Often, the patients experience cough, sputum production and wheezing, as well as repeated bouts of pneumonia, often several times per winter. The airway obstruction is usually irreversible, which means that it persists in spite of treatment with corticosteroids and beta2-agonists. As the disease progresses during many years, the airway obstruction can become very severe, leading to severe dyspnea during both exercise and rest and, eventually, lung failure. At this stage, lung function examinations with spirometri usually reveal a loss of lung capacity by 50% or more. Other severe symptoms often appear at this time as well, such as weight loss, depression and cardiac disease. The mortality risk is high in these patients. The only established pharmaceutical treatment for these patients is anti-cholinergics, which only gives minor effects. Steroids and bronchodilators have minimal beneficial effects.

Asthma is characterized by chronic inflammation in the airways with reversible airway obstruction and bronchial hyperreactivity. In contrast to COPD, asthma is usually treatable with steroids and bronchodilators. However, 10% of asthmatics have severe symptoms in spite of maximum treatment. There is also an overlap between COPD and asthma, often rendering a firm diagnosis difficult to obtain (Chang and Mosenifar, 2007).

COPD in horses (also known as heaves, broken wind, alveolar emphysema and equine asthma) is characterized by inflammation in the airways. It can be caused by dusty or mouldy hay, dust and moulds in bedding, or pollens, dust and other irritants in the environment, but the cause is often unknown. The horses show symptoms like coughing, increased respiration, laboured breathing and yellow nasal discharge. The symptoms range in severity from mild, to so severe that the horse appears listless, has difficulty breathing and develops a muscular “heave line” along the horse's barrel from taking a double exhalation (The Columbia Encyclopedia, 2007). COPD in horses is often treated with β₂-agonists but the bronchorelaxing effect by these drugs is poor (Törneke and Ingvast-Larson, 1999).

There have also been reports about obstructive pulmonary diseases, mainly asthma, in other animals such as cats and dogs. As in humans, these animals get an obstruction of the airways when the bronchi fill up with mucous and go into spasms (bronchoconstriction). It is far more common in cats than dogs, and particularly in Siamese and Himalayan cat breeds (AnimalHospitals-USA, 2007).

Clearly, there are many individuals with COPD and asthma who urgently need better treatments for their disease.

In U.S. Pat. No. 6,063,363 is disclosed a method of treating upper respiratory tract infections with potassium salts, including potassium iodide and bromide. The potassium salt is introduced into the lungs and/or the nasal area and/or the oral cavity as a liquid solution, nasal spray, etc. However, the effect on the upper respiratory tract infections is said to be caused by the potassium cation saturation of the cells and tissues involved in upper respiratory tract infection. The anions of the administrated salts are of no importance in the treatment. Furthermore, U.S. Pat. No. 6,063,363 discloses treatment of infection and not of bronchoconstriction.

In WO 00/36915 is disclosed a method of treating chronic obstructive airway disease by administering an osmotically active compound such as a salt, including potassium iodide and potassium bromide, sugar, sugar alcohol or organic osmolyte to the afflicted airway surface. The osmotically active compound is administered to the airways in order to increase the volume of the liquid on airway surfaces. Iodine salts are disclosed, not elemental iodine, and no bronchorelaxing effect is reported.

U.S. Pat. No. 4,612,122 discloses a process for removing heavy metal ions from the blood comprising passing the blood along an anisothropic membrane which allows the metal ions to diffuse through to a mass of ion-capturing means. The process is used for removing toxic quantities of heavy metal ions from the bloodstream.

In U.S. Pat. No. 6,177,411 a method for treating patients afflicted with heavy metal poisoning is disclosed. The method includes administering mesna (sodium 2-mercaptoethene sulfonate) or dimesna (the disulfide-dimer of mesna) to the patient. Mesna or dimesna works by substituting a thiol moiety for a hydroxy or aquo moiety and thus a more water soluble heavy metal is obtained.

### OBJECTS OF THE INVENTION

It is an object of the present invention to provide a method and a means for producing bronchorelaxation in a human or an animal lung affected by airway obstruction.

Another object of the present invention is to provide a method for producing bronchorelaxation in a human or an animal with chronic obstructive pulmonary disease.

A further object of the present invention is to provide a method for producing bronchorelaxation in a human or an animal with asthma.

An additional object of the present invention is to provide an agent and the use thereof for the treatment of airway obstruction, chronic obstructive pulmonary disease and/or asthma in a human or an animal.

Further objects of the invention will become evident from the following summary of the invention, preferred embodiments and the appended claims.

### SUMMARY OF THE INVENTION

According to the present invention is disclosed a method for producing bronchorelaxation in the lungs of a human or an animal affected by airway obstruction, comprising administration to the body of said human or animal a pharmacologically effective amount of an agent capable of binding mercury in elemental, ionic and/or organic form present in the body to enhance its excretion via the feces and/or the urine.

According to the present invention is also disclosed a method for producing bronchorelaxation in the lungs of a human or an animal affected by airway obstruction, comprising administration to the intestine of said human or animal a pharmacologically effective amount of an agent capable of binding mercury in elemental, ionic and/or organic form present in the intestinal lumen to enhance its excretion via the feces and/or the urine.

According to the present invention is also disclosed a method for producing bronchorelaxation in the lungs of a human or an animal with chronic obstructive pulmonary disease (COPD) and/or asthma, comprising administration to the body of said human or animal of a pharmacologically effective agent capable of binding mercury in elemental, ionic and/or organic form present in the body to enhance its excretion via the feces and/or the urine.

According to the present invention is also disclosed a method for producing bronchorelaxation in the lungs of a human or an animal with chronic obstructive pulmonary disease (COPD) and/or asthma, comprising administration to the intestine of said human or animal of a pharmacologically effective agent capable of binding mercury in elemental, ionic and/or organic form present in the intestinal lumen to enhance its excretion via the feces and/or the urine.

According to the present invention is also disclosed a corresponding use of the agent capable of binding mercury in elemental, ionic and/or organic form and its use for the manufacture of a medicament for the treatment of airway obstruction, chronic obstructive pulmonary disease (COPD) and/or asthma in a human or an animal.

According to the present invention is also disclosed an agent for use in the method capable of forming a water-soluble chelate with mercury(I) and/or mercury(II) ion or a water-insoluble precipitate with mercury(I) and/or mercury(II) ion.

According to the present invention is also disclosed an agent for use in the method capable of binding to an organic mercury species, wherein the organic mercury species in particular is selected from methylmercury chloride, methylmercury cysteinylglycine, methylmercury cysteine and methylmercury glutathione.

According to the present invention is also disclosed an agent for use in the method comprising a polythiol resin or an aliphatic thiol residue, preferably an aliphatic vicinal dithiol residue, preferred are ethylenediaminetetraacetic acid (EDTA), 2,3-dimercapto-1-propanol (BAL), meso-2,3-dimercaptosuccinic acid (DMSA), D-penicillamine and 2,3-dimercaptopropane-1-sulfonate (DMPS).

According to the present invention is also disclosed an agent for use in the method comprising a ureasulfonamide polymer resin, dithiocarbamate-incorporated monodispere polystyrene microspheres or N,N-bis(2-hydroxy-ethyl)glycine on polystyrene divinyl benzene.

Without whishing to be bound by theory it is believed that organic mercury is an important cause of bronchoconstriction. Our observation, which is based on Example 1, that intake of iodinated activated charcoal can give a substantial bronchorelaxation, can be explained in the following way. Iodinated activated charcoal has been used for many years to adsorb mercury from air and various gases. It is possible that when this compound is present in the intestine it binds organic mercury compounds, such as methylmercury, that have been excreted in the bile. Adsorption of organic mercury compounds by iodinated activated charcoal breaks the enterohepatic recirculation of these compounds and lowers the body's organic mercury load. However, there might be another way for iodinated activated charcoal to increase the excretion of organic mercury compounds. Iodine is not firmly bound to the carbon surface, and some elemental iodine can be expected to be released into the intestinal fluid. The free elemental iodine in the intestine reacts with organic mercury, e.g. methylmercury, secreted into the bile to form mercury iodide (Hgl₂). Since mercury salts usually are poorly absorbed from the intestine (˜7% of an oral dose), most of the Hgl₂ is excreted in the feces. Thus, iodinated activated charcoal may lead to an enhanced excretion of organic mercury species by two mechanisms:

1. Direct reaction of organic mercury compounds with iodinated activated charcoal,

2. Elemental iodine released from the iodinated activated charcoal into the intestinal lumen may react with organic mercury compounds. This transforms readily absorbed organic mercury compounds to poorly absorbed mercury iodide, which is excreted by the feces.

In this specification the term iodine refers to elemental iodine, I₂, and the term iodine on activated charcoal refers to iodinated activated charcoal.

According to a first preferred aspect of the invention iodinated activated charcoal is administered to the intestine of a human or an animal in need of bronchorelaxation, in a pharmaceutically acceptable form, in particular in form of a tablet or capsule comprising elemental iodine on activated charcoal capable of binding present in the body mercury in elemental, ionic and/or organic form and, optionally, a bromide salt.

According to a second preferred aspect of the invention iodinated activated charcoal is administered to the intestine of a human or an animal with chronic obstructive pulmonary disease (COPD) and/or asthma, in a pharmaceutically acceptable form, in particular in form of a tablet or capsule comprising elemental iodine on activated charcoal capable of binding present in the intestinal lumen mercury in elemental, ionic and/or organic form and, optionally, a bromide salt.

According to a third preferred aspect of the invention is disclosed the use of an agent capable of binding mercury in elemental, ionic and/or organic form present in the body of a patient or animal for excretion via feces or urine for the manufacture of a medicament for the treatment of airway obstruction in said patient or animal; the use of an agent capable of binding mercury in elemental, ionic and/or organic form present in the body of a patient or animal for excretion via feces or urine for the manufacture of a medicament for the treatment of chronic obstructive pulmonary disease in said patient or animal; the use of an agent capable of binding mercury in elemental, ionic and/or organic form present in the body of a patient or animal for excretion via feces or urine for the manufacture of a medicament for the treatment of asthma in said patient or animal; the use of an agent capable of binding mercury in elemental, ionic and/or organic form present in the intestinal lumen of a patient or animal for excretion via feces for the manufacture of a medicament for the treatment of airway obstruction in said patient or animal; the use of an agent capable of binding mercury in elemental, ionic and/or organic form present in the intestinal lumen of a patient or animal for excretion via feces for the manufacture of a medicament for the treatment of chronic obstructive pulmonary disease in said patient or animal; the use of an agent capable of binding mercury in elemental, ionic and/or organic form present in the intestinal lumen of a patient or animal for excretion via feces for the manufacture of a medicament for the treatment of asthma in said patient or animal. In such use the agent capable of binding mercury is preferably elemental iodine on activated charcoal (iodinated activated charcoal) and optionally is combined with a bromide salt, in particular an alkali bromide. Furthermore, in such use, the agent capable of binding mercury is preferably elemental iodine on an inorganic carrier that is substantially insoluble (<0.1 mg/L) in an aqueous fluid of pH from 6.0 to 8.0. It is preferred for the inorganic carrier to be selected from diatomaceous earth, silica, silicate, aluminum oxide, basic aluminum oxide, titanium dioxide, iron(III)oxide and mixtures thereof.

According to a preferred embodiment of the invention the agent capable of binding mercury is elemental iodine on or in an polymeric organic carrier that is substantially insoluble (<0.1 mg/L) in an aqueous fluid of pH from 5.0 to 8.5, in particular from 6.0 to 8.0, in particular of about 7.0. The polymeric organic carrier is preferably selected from cross-linked starch and microporous organic polymer, in particular microporous polystyrene and microporous polycarbonate.

According to a still further preferred aspect of the invention is disclosed the use of activated charcoal for reducing the amount of elemental mercury dissolved in an aqueous media by 99% or more, in particular by 99.9% or more. This reduction can be obtained within 1 h at a temperature about 40° C. It is preferred for the aqueous media to comprise gastric fluid. It is also preferred for the activated charcoal to comprise elemental iodine.

The invention will now be described in more detail by reference to preferred but not limiting embodiments.

### DESCRIPTION OF PREFERRED EMBODIMENTS OF THE INVENTION

### Example 1

### Administration of Iodine on Activated Charcoal

A male Caucasian, born 1935, had been smoking cigarettes daily for many years, but quit about 10 years ago. The decision to quit smoking was caused by increasing problems from the airways, with repeated episodes of pneumonia and airway obstruction. These symptoms were usually treated with antibiotics, steroids and bronchodilators. The COPD diagnose was first suggested in June 2000.

After that, the airway symptoms increased considerably, with month-long episodes of cough and exercise-induced dyspnea. Spirometric evaluation some years later showed a Forced Expiratory Volume in one second, FEV₁, of 1.44 L, corresponding to 49.7% of his reference value, and a Peak Expiratory Flow, PEF, of 282 L/min, corresponding to 60.1% of his reference value.

In the following months the situation continued to worsen with loss of appetite, reduction of weight and severe dyspnea during rest, in spite of maximum treatment with anti-cholinergics, steroids and bronchodilators. The patient now felt desperately ill, and questioned how long he would be able to survive.

The severity of his condition prompted the patient to look for alternative treatments. When ingesting iodinated activated charcoal, in form of rods (1×1×5 mm) of activated charcoal comprising about 5% iodine by weight, I₂, (Sigma-Aldrich, Inc.) the patient experienced immediate relief of the airway obstruction and dyspnea. A few days intake of 1-2 g iodinated activated charcoal twice per day (equivalent of 50-100 mg iodine twice per day), suspended in yoghurt, produced a dramatic increase in lung function and stamina, and completely removed the dyspnea. Sputum production was also considerably reduced. A renewed spirometric evaluation a few months later confirmed the subjective improvements (FEV₁=2.79 L, corresponding to 97.1% of his reference value and PEF=427 L/min, corresponding to 92.5% of his reference value).

The patient has continued to regularly take the combination for more than a year and still experiences the full benefit of it. A temporary discontinuation of the intake for a few days led to the reappearance of many of the symptoms. However, they quickly disappeared upon the resumption of the intake of iodinated activated charcoal. The patient now lives a normal life, being able to pursue gardening, cycling and even to play an occasional game of badminton.

To test the bronchorelaxing effect of activated charcoal without addition of iodine, the same patient took 5 g of iodine-free activated charcoal (Medikol, Selena Fournier) for a few days. However, no bronchorelaxing effect was observed, instead his condition worsened.

To clarify if iodine in itself has bronchorelaxing properties, 50 mg elemental iodine (Sigma-Aldrich, Inc.), placed in a gelatin capsule, was taken by the same patient for a few days. However, no distinct bronchorelaxing effect was observed.

### Example 2

### Tablets Comprising Iodine on Activated Charcoal

Tablets comprising iodine on activated charcoal were compressed in a conventional tabletting machine from 500 mg iodinated activated charcoal (Sigma-Aldrich, Inc.) mixed with 122 mg lactose monohydrate, 6 mg magnesium stearate and 122 mg sodium methyl cellulose to form a 750 mg tablet comprising about 25 mg iodine. Optionally, sodium bromide (0.5-5% w/w) can be added to the tabletting mixture.

### Example 3

### Capsules Comprising Iodine on Activated Charcoal and Sodium Bromide

Capsules comprising iodine on activated charcoal and sodium bromide were manufactured by mixing 350 mg iodinated activated charcoal (Sigma-Aldrich, Inc.) with 5 mg sodium bromide. Gelatin capsules were filled with the mixture in a conventional capsule filling machine to form capsules containing about 17 mg iodine.

### Example 4

### Capsules Comprising Iodine on Activated Charcoal and Sodium Bromide—for Fast Release in the Stomach

Capsules comprising iodine on activated charcoal and sodium bromide were manufactured by mixing 350 mg iodinated activated charcoal (Sigma-Aldrich, Inc.) with 5 mg sodium bromide. Pullulan capsules were filled with the mixture in a conventional capsule filling machine to form capsules containing about 17 mg iodine.

### Example 5

### Absorption by Activated Charcoal of Hg(0) in Water

A solution of Hg(0) in water was prepared by placing a droplet of mercury in a 250 ml glass beaker, adding 150 mL of distilled water, and stirring for 1 h at 40° C. 100 mL of the aqueous phase was decanted into another 250 mL glass beaker and a sample (30 mL) was withdrawn. Iodinated activated charcoal (0.7 g) was added to the aqueous phase and the suspension was stirred at 40° C. and samples (30 mL) each were taken at 30 min and 60 min. The samples were filtered into glass tubes provided with polypropylene stoppers, 2% nitric acid (0.6 ml) was added to each sample, and the samples sent for analysis. Hg²⁺, mg/mL: 0.075 prior to addition of iodine on charcoal; 0.0055 at 30 min after addition of nitric acid; <0.0001 at 60 min after addition of nitric acid. Thus, 1 h exposure to 1 g of iodinated activated charcoal/100 mL water removed 99.9% by weight of Hg(0) from the solution.

### REFERENCES

Clarkson, T. W. The Toxicology of Mercury. Critical Reviews in Clinical Laboratory Sciences 1997 34(3):369-403.
Suzuki, T., Shishido, S. and Urushiyama, K. Mercury in Cigarettes. Tohoku Journal of Experimental Medicine 1976 119(4):353-356.
Clarkson, T. W. The Three Modern Faces of Mercury. Environmental Health Perspectives 2002 110:11-23.
Virtanen, J. K., Rissanen, T. H., Voultilainen, S. and Tuomainen, T-P. Mercury as a Risk Factor for Cardiovascular Diseases. Journal of Nutritional Biochemistry 2007 18:75-85.
Norseth, T. and Clarkson, T. W. Intestinal Transport of ²⁰³Hg-Labeled Methyl Mercury Chloride. Archives of Environmental Health 1971 22:568-577.
Clarkson T. W., Small, H. and Norseth, T. Excretion and Absorption of Methyl Mercury After Polythiol Resin Treatment. Archives of Environmental Health 1973 26:173-176.
Goyer, R. A., Cheman, M. G., Jones, M. M. and Reigart, J. R. Role of Chelating Agents for Prevention, Intervention, and Treatment of Exposures to Toxic Metals. Environmental Health Perspectives 1995 103(11):1048-1052.
Senkal, B. F., and Yavuz, E. Ureasulfonamide Polymeric Sorbent for Selective Mercury Extraction. Chemical Monthly 2006 137:929-934.
Denizli, A., Kesenci, K., Arica, Y. and Piskin, E. Dithiocarbamate-incorporated monosize polystyrene microspheres for selective removal of mercury ions. Reactive & Functional Polymers 2000 44:235-243.
Dev, K. and Rao, G. N., Preparation and Analytical Properties of a Chelating Resin Containing Bicine Groups. Talanta 1995 42(4):591-596.
Greenwood, N. N. and Earnshaw, A. Chemistry of the Elements. Pergamon Press, 1984. Carbo Tech-Aktivkohlen GmbH, Franz-Ficher-Weg 61, D-45307 Germany.
Mannino, D. M. and Buist, S. A. Gobal burden of COPD: risk factors, prevalence, and future trends. Lancet 2007 370:765-73.
Anderson, R. H., Ramyani, G., Strachan, D. P., and Limb, E. S. 50 years of Asthma: UK trends from 1955 to 2004. Thorax 2007 62:85-90.
Chang, J. and Mosenifar, Z. Differentiating COPD from Asthma in Clinical Practice. Journal of Intensive Care Medicine 2007 22:300-309.
Törneke, K. and Ingvast-Larson, C. Beta2-agonister vid behandling av COPD på häst. Svensk Veterinärtidning 1999 51(1):13-16.
The Columbia Encyclopedia, 6^{th} Ed., Columbia University Press, USA, http://www.encyclopedia.comjdoc/1E1-heaves.html, accessed Nov. 19, 2007.
AnimalHospitals-USA, http://www.animalhospitals-usa.com/dogs/asthma.html, accessed Nov. 19, 2007.

1-37. (canceled)

38. A method of producing bronchorelaxation in the lungs of a human or animal affected by airway obstruction, comprising administration to the intestine of said human or animal of a pharmacologically effective amount of an agent which binds mercury present in the body in organic form, and thereby enhance mercury excretion via feces or urine or both.

39-44. (canceled)

45. The method of claim 38, wherein the agent binds to a methylmercury species.

46. The method of claim 45, wherein the methylmercury species is selected from the group consisting of methylmercury chloride, methylmercury cysteinylglycine, methylmercury cysteine and methylmercury glutathione.

47. The method of claim 45 wherein the agent comprises a polythiol resin.

48. The method of claim 45, wherein the agent comprises a ureasulfonamide polymer resin, dithiocarbamate-incorporated monodisperse polystyrene microspheres or n,/i-bis(2-hydroxy-ethyl)glycine on polystyrene divinyl benzene.

49. The method of claim 45, wherein the agent comprises an aliphatic vicinal thiol residue.

50. (canceled)

51. The method of claim 49, wherein the agent is selected from the group consisting of ethylenediaminetetraacetic acid (EDTA), 2,3-dimercapto-1-propanol (BAL), meso-2,3-dimercaptosuccinic acid (DMSA), D-penicillamine and 2,3-dimercaptopropane-1-sulfonate (DMPS).

52. The method of claim 38, wherein the agent comprises elemental iodine on activated charcoal (iodinated activated charcoal).

53-54. (canceled)

55. The method of claim 38, wherein the administration is in form of a tablet.

56. The method of claim 55, wherein the tablet comprises a bromide salt.

57. The method of claim 55, wherein the tablet comprising disintegrant for fast release of the tablet contents in the stomach.

58. The method of claim 38, wherein administration is in form of a capsule.

59. The method of claim 58, a wherein the capsule comprises a bromide salt.

60. The method of claim 58, wherein the capsule is a gelatin capsule.

61. The method of claim 58, wherein the capsule is a pullulan capsule.

62. The method of claim 38, wherein the airway obstruction is caused by chronic obstructive pulmonary disease.

63. The method of claim 38, wherein the airway obstruction is caused by asthma.

64. The method of claim 38, wherein the agent comprises a polythiol resin.

65. The method of claim 64, wherein the agent comprises a ureasulfonamide polymer resin, dithiocarbamate-incorporated monodisperse polystyrene microspheres or n,/i-bis(2-hydroxy-ethyl)glycine on polystyrene divinyl benzene.

66. The method of claim 38, wherein the agent comprises an aliphatic vicinal thiol residue.

67. The method of claim 66, wherein the agent is selected from the group consisting of ethylenediaminetetraacetic acid (EDTA), 2,3-dimercapto-1-propanol (BAL), meso-2,3-dimercaptosuccinic acid (DMSA), D-penicillamine and 2,3-dimercaptopropane-1-sulfonate (DMPS).
